SEQUENCE LISTING (10 sequences)

SEQ ID NO: 1 - protein, 5 aa, synthetic construct
  REGION 1..5: note = Synthetic Construct
AYSVN

SEQ ID NO: 2 - protein, 16 aa, synthetic construct
  REGION 1..16: note = Synthetic Construct
MIWGDGKIVYNSALKS

SEQ ID NO: 3 - protein, 10 aa, synthetic construct
  REGION 1..10: note = Synthetic Construct
DGYYPYAMDN

SEQ ID NO: 4 - protein, 15 aa, synthetic construct
  REGION 1..15: note = Synthetic Construct
RASKSVDSYGNSFMH

SEQ ID NO: 5 - protein, 7 aa, synthetic construct
  REGION 1..7: note = Synthetic Construct
LASNLES

SEQ ID NO: 6 - protein, 9 aa, synthetic construct
  REGION 1..9: note = Synthetic Construct
QQNNEDPRT

SEQ ID NO: 7 - protein, 117 aa, synthetic construct
  REGION 1..117: note = Synthetic Construct
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 111 aa, synthetic construct [sequence omitted]

SEQ ID NO: 9 - protein, 445 aa, synthetic construct
  REGION 1..445: note = Synthetic Construct
  SITE 1: note = MISC_FEATURE - Xaa at position 1 is Q, pE, or absent
  SITE 444: note = MISC_FEATURE - Xaa at position 444 is G or absent
  SITE 445: note = MISC_FEATURE - Xaa at position 445 is K or absent
XVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 10 - protein, 218 aa, synthetic construct
  REGION 1..218: note = Synthetic Construct
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0425578 A1
(43) Pub. Date: Dec. 26, 2024

(54) IL-13 INHIBITORS FOR THE TREATMENT OF PRURIGO NODULARIS

(71) Applicant: DERMIRA, INC., Indianapolis, IN (US)

(72) Inventors: Renata Gontijo LIMA, Ponte Vedra, FL (US); Kimberly Mung Chee SIU, Jupiter, FL (US)

(21) Appl. No.: 18/691,331

(22) PCT Filed: Sep. 14, 2022
(86) PCT No.: PCT/US2022/076387

§ 371 Date: Mar. 12, 2024

Related U.S. Application Data
(60) Provisional application No. 63/244,427, filed on Sep. 15, 2021.

(51) Int. Cl. C07K 16/24; A61K 39/00; A61P 17/04

(52) U.S. Cl. CPC C07K 16/244; A61P 17/04; A61K 2039/505; C07K 2317/24

(57) ABSTRACT

Provided herein are methods and uses of IL-13 inhibitor (e.g., anti-IL-13 antibodies) for treating prurigo nodularis or reducing pruritis associated with prurigo nodularis.

SEQUENCE LISTING

The present application is being filed along with a Sequence Listing in ST.26 XML format. The Sequence Listing is provided as a file titled “X23072_SequenceListing” created Sep. 13, 2022 and is 15 kilobytes in size. The Sequence Listing information in the ST.26 XML format is incorporated herein by reference in its entirety.

### FIELD

The present invention relates to methods and uses of interleukin (IL)-13 inhibitors for treating prurigo nodularis or reducing pruritis associated with prurigo nodularis.

### BACKGROUND

Prurigo nodularis (PN) is a chronic inflammatory skin disorder characterized by intensely pruritic, hyperkeratotic nodules (Huang et al., J Am Acad Dermatol. 2020; 83 (6):1559-1565). PN lesions can cause intense itching, bleeding, burning, and stinging sensations. The physical discomfort combined with the potential psychological effects of the disease interfere with everyday activities and significantly impact an individual's quality of life. To date, no treatment for PN has been approved and patients are often refractory to off-label treatments and experience a significant burden on their quality of life (Huang et al., J Am Acad Dermatol. 2020; 83 (6):1559-1565).

Interleukin (IL)-13 is a key mediator of T-helper type 2 (Th2) inflammation and signals through a heterodimeric receptor IL-4Rα/IL-13Ra1. Some evidence suggest that Th2cytokines might play a role in the pathogenesis of PN.

There remains a substantial unmet medical need for effective therapies for treating PN.

### SUMMARY OF INVENTION

Provided herein are methods and uses of an IL-13 inhibitor (e.g., an anti-IL-13 antibody), or pharmaceutical compositions comprising an IL-13 inhibitor (e.g., an anti-IL-13 antibody), for treating prurigo nodularis or reducing pruritis associated with prurigo nodularis.

In one aspect, provided herein are methods of treating prurigo nodularis or reducing pruritus associated with prurigo nodularis in a patient in need thereof, and such methods comprise administering to the patient a therapeutically effective amount of an IL-13 inhibitor. In some embodiments, the IL-13 inhibitor is an anti-IL-13 antibody. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab.

In some embodiments, the anti-IL-13 antibody is administered subcutaneously to the patient. In some embodiments, the anti-IL-13 antibody is administered at a dose of 250 mg to 500 mg. In some embodiments, the anti-IL-13 antibody is administered subcutaneously to the patient at a dose of 250 mg once every two weeks. In some embodiments, the anti-IL-13 antibody is administered subcutaneously to the patient at a dose of 250 mg once every four weeks. In some embodiments, the patient is further treated with a loading dose of 500 mg of the anti-IL-13 antibody. In some embodiments, the loading dose is administered once or twice to the patient. In some embodiments, the loading dose is administered to the patient twice at week 0 (baseline) and week 2. In some embodiments, the patient is treated with the anti-IL-13 antibody for a period of about 16 weeks or more (e.g., about 16 to about 24 weeks).

In some embodiments, the patient has moderate to severe prurigo nodularis. In some embodiments, the patient has (1) a clinical diagnosis of PN for at least 6 months; (2) PN lesions on upper limbs, trunk, and/or lower limbs, at least 20 nodules on the entire body with a bilateral distribution; (3) a IGA PN-S score ≥3 at baseline; and/or (4) an Itch NRS score ≥7 at baseline. In some embodiments, the patient has inadequate response to topical corticosteroids (TCS) or topical calcineurin inhibitors (TCI), or TCS or TCI are medically inadvisable for the patient. In some embodiments, the patient is aged 18 years or older.

In some embodiments, the methods further comprise determining the Itch Numerical Rating Scale (NRS) score of the patient before and after the treatment. In some embodiments, the methods further comprise determining one or more of the following characteristics of the patient: IGA PN-S (Investigator's Global Assessment: Prurigo Nodularis-Stage), IGA PN-A (Investigator's Global Assessment: Prurigo Nodularis-Activity), Skin Pain NRS, nighttime awakenings DSS (Dermatology Sleep Scale), PGI-S-PN (Patient Global Impression of Severity-Prurigo Nodularis), DLQI (Dermatology Life Quality Index), PAS (Prurigo Activity Score), PROMIS (Patient-Reported Outcomes Measurement Information System) Anxiety and Depressive Symptoms, and EuroQol-5D (European Quality of Life-5 Dimensions-5 Levels or EQ-5D-5L) before and after the treatment.

In another aspect, provided herein are an IL-13 inhibitor (e.g., an anti-IL-13 antibody) or pharmaceutical composition comprising an IL-13 inhibitor (e.g., an anti-IL-13 antibody) for use in the treatment of prurigo nodularis or reducing pruritus associated with prurigo nodularis. Also provided herein are uses of an IL-13 inhibitor (e.g., an anti-IL-13 antibody) in the manufacture of a medicament for the treatment of prurigo nodularis or reducing pruritus associated with prurigo nodularis.

### BRIEF DESCRIPTION OF THE DRAWINGS

FIG. 1A is a dot plot showing the IL-13 levels in prurigo nodularis patient samples (PN) and healthy control samples (HC). FIG. 1B is a dot plot showing periostin levels in PN and HC samples. FIG. 1C is a dot plot showing IL-4 levels in PN and HC samples. FIG. 1D is a dot plot showing IL-5 levels in PN and HC samples. FIG. 1E is a dot plot showing IgE levels in PN and HC samples.

FIG. 2 is a schematic diagram of the Phase 3 study design described in Example 2. Footnotes mean:^{a} Participants requiring rescue therapy for PN during the screening period or who are expected to require rescue therapy within 4 weeks following the baseline visit are excluded from the study.^{b} Participants who require use of low to mid-potency TCS or TCI may do so only during the first 2 weeks of the treatment period for ≤3 consecutive days and no more than 5 cumulative days. These participants are be considered treatment failures in the efficacy analyses. ^{c} At Weeks 0 (baseline) and 2, participants in the lebrikizumab treatment arm receive a 500 mg loading dose, and participants in the placebo arm receive a placebo loading dose.^{d} After completion of the Week 16 visit, participants are offered the option of continuing treatment in a separate long-term extension study. Participants who discontinue the study early or choose not to enter the long-term extension study must return for a post-treatment safety follow-up visit approximately 12 weeks after the date of the last study drug injection.

### DETAILED DESCRIPTION

Provided herein are methods and uses of an IL-13 inhibitor (e.g., an anti-IL-13 antibody), or pharmaceutical compositions comprising an IL-13 inhibitor (e.g., an anti-IL-13 antibody), for treating prurigo nodularis or reducing pruritis associated with prurigo nodularis.

In one aspect, provided herein are methods of treating prurigo nodularis or reducing pruritus associated with prurigo nodularis in a patient in need thereof, and such methods comprise administering to the patient a therapeutically effective amount of an IL-13 inhibitor. In some embodiments, the IL-13 inhibitor is an anti-IL-13 antibody. Other IL-13 inhibitors include small molecule inhibitors, antisense oligonucleotides, RNAi agents, aptamers, or peptides that interact with IL-13 or its receptors and decreases or eliminates one or more activities or functions associated with IL-13.

In some embodiments, the patient has moderate to severe prurigo nodularis. In some embodiments, the patient has (1) a clinical diagnosis of PN for at least 6 months; (2) PN lesions on upper limbs, trunk, and/or lower limbs, at least 20 nodules on the entire body with a bilateral distribution; (3) a IGA PN-S score≥3 at baseline; and/or (4) an Itch NRS score≥7 at baseline. In some embodiments, the patient has inadequate response to TCS or TCI, or TCS or TCI are medically inadvisable for the patient. In some embodiments, the patient is aged 18 years or older. In some embodiments, the patient has no prior exposure to dupilumab, tralokinumab, cendakimab, or nemolizumab.

Anti-IL-13 antibodies suitable for use in the methods and uses provided herein have been described previously, e.g., WO2005/062967. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab (CAS No. 953400-68-5). Lebrikizumab is a humanized monoclonal IgG4 antibody that specifically binds IL-13 with high affinity and blocks signaling through the active IL-4Ralpha/IL-13Ralphal heterodimer. The amino acid sequences of lebrikizumab are provided in Table 1. C-terminal clipping of IgG antibodies could occur when one or two C-terminal amino acids are removed from the heavy chain of the IgG antibodies. For example, if a C-terminal lysine (K) is present, it may be truncated or clipped off from the heavy chain. A penultimate glycine (G) may also be truncated or clipped off from the heavy chain as well. Modification of N-terminal amino acid of IgG could also occur. For example, the N-terminal glutamine (Q) or glutamic acid (E) can cyclize into pyro-glutamate (pE) spontaneously. SEQ ID NO: 9 reflects these potential modifications of lebrikizumab heavy chain.

TABLE 1
Lebrikizumab Sequences

| SEQ ID NO: | Description | Sequence |
|---|---|---|
| 1 | Lebrikizumab | AYSVN |
| | HCDR1 | |
| 2 | Lebrikizumab | MIWGDGKIVYNSALKS |
| | HCDR2 | |
| 3 | Lebrikizumab | DGYYPYAMDN |
| | HCDR3 | |
| 4 | Lebrikizumab | RASKSVDSYGNSFMH |
| | LCDR1 | |
| 5 | lebrikizumab | LASNLES |
| | LCDR2 | |
| 6 | Lebrikizumab | QQNNEDPRT |
| | LCDR3 | |
| 7 | Lebrikizumab | VTLRESGPALVKPTQTLTLTCTVSGFSLSAYSVN |
| | heavy chain | WIRQPPGKALEWLAMIWGDGKIVYNSALKSRLTI |
| | variable region | SKDTSKNQVVLTMTNMDPVDTATYYCAGDGYY |
| | (VH) | PYAMDNWGQGSLVTVSS |
| 8 | Lebrikizumab | DIVMTQSPDSLSVSLGERATINCRASKSVDSYGN |
| | light chain | SFMHWYQQKPGQPPKLLIYLASNLESGVPDRFSG |
| | variable region | SGSGTDFTLTISSLQAEDVAVYYCQQNNEDPRTF |
| | (VL) | GGGTK VEIK |
| 9 | Lebrikizumab | Xaa1VTLRESGPALVKPTQTLTLTCTVSGFSLSAYS |
| | heavy chain | VNWIRQPPGKALEWLAMIWGDGKIVYNSALKSR |
| | (HC) | LTISKDTSKNQVVLTMTNMDPVDTATYYCAGDG |
| | | YYPYAMDNWGQGSLVTVSSASTKGPSVFPLAPC |
| | | SRSTSESTAALGCLVKDYFPEPVTVSWNSGALTS |
| | | GVHTFPAVLQSSGLYSLSSVVTVPSSSLGTKTYTC |
| | | NVDHKPSNTKVDKRVESKYGPPCPPCPAPEFLGG |
| | | PSVFLFPPKPKDTLMISRTPEVTCVVVDVSQEDPE |
| | | VQFNWYVDGVEVHNAKTKPREEQFNSTYRVVS |
| | | VLTVLHQDWLNGKEYKCKVSNKGLPSSIEKTISK |
| | | AKGQPREPQVYTLPPSQEEMTKNQVSLTCLVKG |
| | | FYPSDIAVEWESNGQPENNYKTTPPVLDSDGSFF |
| | | LYSRLTVDKSRWQEGNVFSCSVMHEALHNHYTQ |
| | | KSLSLSLXaa2Xaa3 |
| | | wherein Xaal is Q, pE, or absent; Xaa2 is G or absent; |
| | | Xaa3 is K or absent. |
| 10 | Lebrikizumab | DIVMTQSPDSLSVSLGERATINCRASKSVDSYGN |
| | light chain | SFMHWYQQKPGQPPKLLIYLASNLESGVPDRFSG |
| | (LC) | SGSGTDFTLTISSLQAEDVAVYYCQQNNEDPRTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQ |
| | | GLSSPVTKSFNRGEC |

Other exemplary anti-IL-13 antibodies include, but not limited to, IMA-026, IMA-638 (also referred to as, anrukinzumab, QAX-576, CAS No. 910649-32-0), tralokinumab (also referred to as CAT-354, CAS No. 1044515-88-9); cendakimab (also referred to as CC-93538, RPC4046, ABT-308, CAS No. 2151032-62-9), AER-001, ABT-308 (also referred to as humanized 13C5.5 antibody). Examples of such anti-IL-13 antibodies and other inhibitors of IL-13 are disclosed, for example, in WO2008/086395, WO2006/085938, U.S. Pat. Nos. 7,615,213, 7,501,121, 7,935,343, 7,829,090, 7,947,273, WO2007/036745, WO2010/073119, WO2007/045477, WO 2014/165771. In some embodiments, the anti-IL-13 antibody is tralokinumab. In some embodiments, the anti-IL-13 antibody is cendakimab.

The anti-IL-13 antibody can be formulated with suitable carriers or excipients into a pharmaceutical composition that is suitable for administration to patients. For example, the anti-IL-13 antibody, e.g., lebrikizumab, can be formulated in a pharmaceutical composition as described in WO 2013/066866. The pharmaceutical composition can comprise 100 mg, 150 mg, 200 mg, 250 mg, 300 mg, 350 mg, 400 mg, 450 mg, or 500 mg of the anti-IL-13 antibody. In some embodiments, the pharmaceutical composition comprises 250 mg to 500 mg of the anti-IL-13 antibody. In some embodiments, the pharmaceutical composition comprises 250 mg or 500 mg of the anti-IL-13 antibody. In some embodiments, the anti-IL-13 antibody concentration in the pharmaceutical composition is between 100 mg/mL and 150 mg/mL, e.g., 125 mg/mL. The pharmaceutical composition can also comprise a buffer, e.g., 5 mM-40 mM histidine acetate buffer, pH 5.4 to 6.0. In some embodiments, the pharmaceutical composition further comprises a polyol (e.g., sugar) that has a concentration between 100 mM and 200 mM, and/or a surfactant (e.g., polysorbate 20) that has a concentration of 0.01%-0.1%. In one embodiment, the pharmaceutical composition comprises 125 mg/mL of an anti-IL-13 antibody (e.g., lebrikizumab), 20 mM histidine acetate buffer, pH 5.7, 175 mM sucrose and 0.03% polysorbate 20.

The IL-13 inhibitor or a pharmaceutical composition comprising the IL-13 inhibitor can be administered to the patient intravenously, orally, intramuscularly, or subcutaneously.

In some embodiments, the patient is treated with the IL-13 inhibitor (e.g., anti-IL-13 antibody) or a pharmaceutical composition comprising the IL-13 inhibitor (e.g., anti-IL-13 antibody) for a period of about 16 weeks or more (e.g., about 16 to about 24 weeks, about 16 to about 32 weeks, about 16 to about 36 weeks, about 16 to about 48 weeks, about 16 to about 52 weeks, about 16 to about 60 weeks). In some embodiments, the patient is treated with the IL-13 inhibitor (e.g., anti-IL-13 antibody) or a pharmaceutical composition comprising the IL-13 inhibitor (e.g., anti-IL-13 antibody) for about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, or about 60 weeks. In some embodiments, the patient is treated with the IL-13 inhibitor (e.g., anti-IL-13 antibody) or a pharmaceutical composition comprising the IL-13 inhibitor (e.g., anti-IL-13 antibody) for a period of about 16 to about 24 weeks. In some embodiments, the patient is treated with the IL-13 inhibitor (e.g., anti-IL-13 antibody) or a pharmaceutical composition comprising the IL-13 inhibitor (e.g., anti-IL-13 antibody) for a period of about 16 weeks. In some embodiments, the patient is treated with the IL-13 inhibitor (e.g., anti-IL-13 antibody) or a pharmaceutical composition comprising the IL-13 inhibitor (e.g., anti-IL-13 antibody) for a period of about 24 weeks.

In some embodiments, the anti-IL-13 antibody or a pharmaceutical composition comprising the anti-IL-13 antibody is administered subcutaneously to the patient. The anti-IL-13 antibody or a pharmaceutical composition comprising the anti-IL-13 antibody can be administered to the patient at a dosing frequency of about once a week, once every two weeks, once every three weeks, once every four weeks, once every five weeks, once every six weeks, once every seven weeks, or once every eight weeks. In some embodiments, the anti-IL-13 antibody or a pharmaceutical composition comprising the anti-IL-13 antibody is administered to the patient once every two weeks or once every four weeks. In some embodiments, the anti-IL-13 antibody or a pharmaceutical composition comprising the anti-IL-13 antibody is administered subcutaneously to the patient at a dose of 250 mg once every two weeks. In some embodiments, the anti-IL-13 antibody or a pharmaceutical composition comprising the anti-IL-13 antibody is administered subcutaneously to the patient at a dose of 250 mg once every four weeks. In some embodiments, the anti-IL-13 antibody or a pharmaceutical composition comprising the anti-IL-13 antibody is administered subcutaneously to the patient at a dose of 300 mg once every two weeks.

In some embodiments, the patient is treated with a loading dose of the anti-IL-13 antibody, e.g., a loading dose of 500 mg of the anti-IL-13 antibody. The loading dose can be administered a few times to the patient at the beginning of the treatment. For example, a loading dose of 500 mg of the anti-IL-13 antibody can be administered twice at the baseline (week 0) and week 2. After the loading dose, the anti-IL-13 antibody can be administered to the patient at a dose of 250 mg once every two weeks or 250 mg once every four weeks. In some embodiments, after the loading dose, the anti-IL-13 antibody is administered to the patient at a dose of 250 mg once every two weeks. In some embodiments, after the loading dose, the anti-IL-13 antibody is administered to the patient at a dose of 300 mg once every two weeks.

In some embodiments, the anti-IL-13 antibody or a pharmaceutical composition comprising the anti-IL-13 antibody is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). In some embodiments, the subcutaneous administration device is an autoinjector device described in WO 2008/112472, WO 2011/109205, WO 2014/062488, or WO 2016/089864.

Before, during and after the treatment period, the patient can be assessed for one or more characteristics, which determine certain signs, symptoms, features, or parameters that have been associated with prurigo nodularis and that can be quantitatively or qualitatively assessed. Such characteristics include, but are not limited to, Itch NRS, IGA PN-S, IGA PN-A, Skin Pain NRS, nighttime awakenings DSS, PGI-S-PN, DLQI, PAS, PROMIS Anxiety and Depressive Symptoms, and EuroQol-5D (EQ-5D-5L).

Itch Numeric Rating Scale (Itch NRS) is a participant administered, single-item, 11-point horizontal scale anchored at 0 and 10, with 0 representing “no itch” and 10 representing “worst itch imaginable” in adults. Overall severity of a participant's itching is indicated by selecting the number that best describes the worst level of itching in the past 24 hours (Naegeli et al., Int J Dermatol. 2015; 54 (6):715-722; Kimball et al., Br J Dermatol. 2016; 175 (1):157-162; Newton et al., J Patient Rep Outcomes. 2019; 3 (1):42). The Itch NRS can be completed daily by the participant using an eDiary.

Investigator's Global Assessment: Prurigo Nodularis Stage (IGA PN-S) is an investigator-administered, single-item scale for rating the severity of the participant's PN in adults. The IGA PN-S is composed of a 5-point scale ranging from 0 (clear) to 4 (severe), and a score is selected using descriptors that best describe clinical characteristics of number of nodules and their thickness as guidelines for the overall severity assessment. The recall period of this assessment is present time.

Investigator's Global Assessment: Prurigo Nodularis Activity (IGA PN-A) is an investigator-administered, single-item scale for rating the overall activity of the of PN lesions. The IGA PN-A is composed of a 5-point scale ranging from 0 (clear) to 4 (severe), and a score is selected using descriptors based on clinical characteristics of excoriations, crusting, and/or bleeding as guidelines for the overall activity assessment. The number of pruriginous lesions should not be considered for this assessment. The recall period of this assessment is present time.

Skin Pain Numeric Rating Scale (Skin Pain NRS) is a participant administered, 11-point horizontal scale anchored at 0 and 10, with 0 representing “no pain” and 10 representing “worst pain imaginable.” Overall severity of a participant's skin pain is indicated by selecting the number that best describes the worst level of skin pain in the past 24 hours (Newton et al., J Patient Rep Outcomes. 2019; 3 (1):42). The Skin Pain NRS can be completed daily by the participant using an eDiary.

Dermatology Sleep Scale (DSS) is a participant administered 3 item questionnaire in adults developed to assess the impact of itch on sleep including difficulty falling asleep, frequency of waking, and difficulty getting back to sleep last night. Participant's rate their difficulty falling asleep and difficulty getting back to sleep, items 1 and 3, respectively, using a 5-point Likert type scale with response options ranging from 0 “not at all” to 4 “very difficult.” Participants report their frequency of waking last night, item 2, by selecting the number of times they woke up each night, ranging from 0 to 29 times. The DSS is designed to be completed each day with respondents thinking about sleep “last night.” Each item is scored individually. The DSS assessment can be completed daily by the participant using an eDiary.

Prurigo Activity Score (PAS) is a 7-item physician-assessed questionnaire designed to monitor the distribution and activity of chronic prurigo lesions (Polking et al., J Eur Acad Dermatol Venereol. 2018; 32 (10):1754-1760).

Dermatology Life Quality Index (DLQI) is a simple, participant-administered, 10-item, validated, QoL questionnaire in adults that covers 6 domains including symptoms and feelings, daily activities, leisure, work and school, personal relationships, and treatment. The recall period of this scale is over the “last week.” Response categories include “not at all,” “a little,” “a lot,” and “very much,” with corresponding scores of 0, 1, 2, and 3, respectively, and unanswered (or “not relevant”) responses scored as 0. Scores range from 0 to 30 with higher scores indicating greater impairment of QoL. A DLQI total score of 0 to 1 is considered as having no effect on a participant's health-related QOL (Hongbo et al., J Invest Dermatol. 2005; 125 (4):659-664), and a 4-point change from baseline is considered as the minimal clinically important difference threshold (Khilji et al., Br J Dermatol. 2002; 147; Basra et al., Dermatology. 2015; 230 (1):27-33).

Patient Global Impression of Severity-Prurigo Nodularis (PGI-S-PN) is a participant-administered single item assessment asking the patient how they would rate their overall PN symptoms over the past 24 hours in adults. The 5 categories of responses range from “no symptoms” to “severe.”

Patient-Reported Outcomes Measurement Information System (PROMIS) is a set of person-centered measures that evaluates and monitors physical, mental, and social health in adults and children. The PROMIS® measures used in this study include Anxiety and Depression short forms, which assess the patients' symptoms over the previous week. It can be used with the general population and with individuals living with chronic conditions.

The PROMIS Anxiety Short Form v1.0—Anxiety 8a is a participant administered questionnaire that assesses the following items in adults: self-reported fear (fearfulness, panic); anxious misery (worry, dread); hyperarousal (tension, nervousness, restlessness), and somatic symptoms related to arousal (racing heart, dizziness) (PROMIS Anxiety 2019, Published Mar. 1, 2019. Accessed Mar. 8, 2021. Available at https://www.healthmeasures.net/images/PROMIS/manuals/PROMIS_Anxiety_Scoring_Manual.pdf). Each question has 5 response options, with scores ranging from 1 to 5. The total scores range from 8 to 40, with the higher score indicating a higher level of anxiety. The adult self-report assesses anxiety “in the past 7 days.”

The PROMIS Depression Short Form v1.0-Depression 8a is a participant administered questionnaire that assesses the following items in adults: self-reported negative mood (sadness, guilt); views of self (self-criticism, worthlessness); social cognition (loneliness, interpersonal alienation), and decreased positive affect and engagement (loss of interest, meaning, and purpose) (PROMIS Depression 2019, Published Feb. 28, 2019. Accessed Mar. 8, 2021. Available at https://www.healthmeasures.net/images/PROMIS/manuals/PROMIS_Depression_Scoring_Manual.pdf). Somatic symptoms (such as changes in appetite or sleeping patterns) are not included. This helps eliminate potential confounding effects of these items when assessing participants with co-morbid physical conditions. Each question has 5 response options, with scores ranging from 1 to 5. The total scores range from 8 to 40, with the higher score indicating a higher level of depression. The adult self-report assesses depression “in the past 7 days.”

The European Quality of Life-5 Dimensions-5 Levels (EuroQol-5D-5L or EQ-5D-5L) is a participant-administered, 5 questions plus 1 visual analog scale (VAS) standardized measure of health status in adults that provides a simple, generic measure of health for clinical and economic appraisal. The EQ-5D-5L consists of 2 components: a descriptive system of the respondent's health and a rating of his or her current health state using a 0 to 100 mm VAS (20 cm). The descriptive system comprises the following 5 dimensions: mobility, self care, usual activities, pain/discomfort, and anxiety/depression. Each dimension has 5 levels: no problems, slight problems, moderate problems, severe problems, and extreme problems. The respondent is asked to indicate his or her health state by ticking (or placing a cross) in the box associated with the most appropriate statement in each of the 5 dimensions. It should be noted that the numerals 1 to 5 have no arithmetic properties and should not be used as an ordinal score. The EQ-5D-5L health states, defined by the EQ-5D-5L descriptive system, may be converted into a single summary index by applying a formula that essentially attaches values (also called weights) to each of the levels in each dimension. The VAS records the respondent's self-rated health on a vertical VAS where the endpoints are labeled “best imaginable health state” and “worst imaginable health state.” This information can be used as a quantitative measure of health outcome (Herdman et al., Qual Life Res. 2011; 20 (10): 1727-1736; EuroQol Group, EQ-5D-5L User Guide. Version 2.1. April 2015. Accessed: Jan. 14, 2021. Available at https://euroqol.org/wp-content/uploads/2016/09/EQ-5D-5L_UserGuide_2015.pdf). The self-rated health status captured by EQ-5D-5L relates to the participant's situation at the time of completion. No attempt is made to recall health status over the preceding days or weeks (EuroQol Group 2015).

The described characteristics can be measured at baseline and at one or more time points after administration of the IL-13 inhibitor (e.g., anti-IL-13 antibody) or a pharmaceutical composition comprising the IL-13 inhibitor (e.g., anti-IL-13 antibody). For example, they may be measured at the end of week 1, week 2, week 3, week 4, week 5, week 6, week 7, week 8, week 9, week 10, week 11, week 12, week 13, week 14, week 15, week 16, or longer after the initial treatment with the IL-13 inhibitor (e.g., anti-IL-13 antibody) or a pharmaceutical composition comprising the IL-13 inhibitor (e.g., anti-IL-13 antibody). The difference between the value at a particular time point following initiation of treatment and the value at baseline is used to establish whether there has been an improvement (e.g., a reduction) in the characteristics.

In another aspect, provided herein are an IL-13 inhibitor (e.g., anti-IL-13 antibody) or pharmaceutical composition comprising an IL-13 inhibitor (e.g., anti-IL-13 antibody) for use in the treatment of prurigo nodularis or reducing pruritus associated with prurigo nodularis. Also provided herein are uses of an IL-13 inhibitor (e.g., anti-IL-13 antibody) in the manufacture of a medicament for the treatment of prurigo nodularis or reducing pruritus associated with prurigo nodularis.

As used herein, the term “a,” “an,” “the” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Exemplary embodiments of antibodies of the present disclosure also include antibody fragments or antigen-binding fragments, which comprise at least a portion of an antibody retaining the ability to specifically interact with an antigen such as Fab, Fab′, F(ab′)2, Fv fragments, scFv, scFab, disulfide-linked Fvs (sdFv), a Fd fragment and linear antibodies.

The term “anti-IL-13 antibody”, as used herein, refers to an antibody that specifically binds human IL-13. In some embodiments, an anti-IL-13 antibody binds human IL-13 with a dissociation constant (KD) of ≤1μM, ≤100 nM, ≤10 nM, ≤1 nM, ≤0.1 nM, or ≤0.01 nM, (e.g., 10-8 M or less, or 10-9 M or less).

The term “baseline”, as used herein, means prior to or at the time of administration of the first dose of the anti-IL-13 antibody (week 0) or a pharmaceutical composition comprising the anti-IL-13 antibody.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

An “effective amount” of an agent refers to an amount effective, at dosages and for periods of time necessary, to achieve the desired treatment result (e.g., achieve an improvement of ≥4 points from baseline in Itch NRS or IGA PN-S score of 0 or 1).

The term “IL-13”, as used herein, refers to any interleukin-13 isoform from human, unless otherwise indicated. The term encompasses “full-length”, unprocessed IL-13 as well as any form of IL-13 that results from processing in the cell. The term also encompasses naturally occurring variants of IL-13, e.g., splice variants or allelic variants. The amino acid sequences of exemplary human IL-13 are known, e.g., NCBI Accession Nos. NP_002179.2, NP_001341920.1, NP_001341921.1, NP_001341922.1; UniProtKB Accession No. P35225.

The term “IL-13 inhibitor”, as used herein, refers to an agent that interacts with IL-13 or its receptors and decreases or eliminates one or more activities or functions associated with IL-13.

The term “inadequate response” as used herein refers to inability to achieve good disease control of prurigo nodularis (e.g., not able to achieve an improvement of ≥4 points from baseline in Itch NRS or IGA PN-S score of 0 or 1) after use of the treatment for the duration recommended by the product prescribing information.

The term “loading dose” means a dose of a drug given at the beginning of a course of treatment that is higher than the dose given subsequently and each dose given for the remainder of the treatment, which is referred to as a “maintenance dose” or “subsequent dose”. Typically, a loading dose is administered once or twice. After administration of the loading dose or loading doses, a maintenance dose or subsequent dose is administered thereafter, typically at regular intervals, for the remainder of the course of treatment.

The term “patient”, as used herein, refers to a human patient.

The term “topical corticosteroid” or “TCS”, as used herein, includes Group I, Group II, Group III and Group IV topical corticosteroids. According to the Anatomical Therapeutic Chemical (ATC) Classification System of World Health Organization, the corticosteroids are classified as weak (Group I), moderately potent (Group II) and potent (Group III) and very potent (Group IV), based on their activity as compared to hydrocortisone. Group IV TCS (very potent) are up to 600 times as potent as hydrocortisone and include clobetasol and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

The term “topical calcineurin inhibitor” or “TCI”, as used herein, includes pimecrolimus, tacrolimus, and other inhibitors that suppress calcineurin activities and can be topically applied to a patient's skin.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. The IL-13 Level is Elevated in Prurigo Nodularis Patient Samples

The IL-13, IL-4, IL-5, IgE, and periostin levels in prurigo nodularis (PN) patient plasma samples and healthy control (HC) samples were measured and compared.

Levels of IL-13 were assessed by Quanterix single molecule array (Simoa) bead-based 2.0 assays (Lexington, MA) on the Simoa HD-X analyzer and all samples, standards and controls were run in calibrator diluent (provided with kit; 102732). Conjugated paramagnetic beads, biotinylated detection antibodies, and associated buffers were resuspended and loaded onto the Simoa HD-X instrument according to manufacturer protocol. Samples were diluted in 96-well plates and then loaded onto the instrument for automated analysis. The Simoa HD-X mixed each sample with beads, incubated, and added detection antibodies, building an immunocomplex on the bead itself. These bead complexes (only containing up to one target-specific immunocomplex) were then pushed over a specialized disk to separate out individual beads into thousands of femtoliter sized wells on the disk and finally read by the Simoa HD-X giving results down to the fg/ml level in assayed fluids. The standard curve for IL-13 run from 15 pg/ml to 0.001 pg/ml. Lowest level of quantitation (LLoQ) is 0.003 pg and lowest level of detection (LoD) is 0.001 pg/ml with the minimum required dilution (MRD) being 2-fold (1 part plasma to 1 part calibrator diluent) for ACD-A plasma (n=2). All samples assessed fell above LLoQ except for PN02, PN06, HC 27, HC 29, and HC30, which fell between LLOQ and LOD and were reported at value reflected by their signal but should be assessed with additional caution due to increase potential for error below LLoQ. The reported IL-13 assay values were log 2 converted. The log 2 converted values were used to calculate mean levels and fold-change between PN and HC samples. The log2 fold change was then converted to linear fold change (a 4-fold change with p=0.002).

IL-4 and IL-5 were measured with S-PLEX Human IL-4 and IL-5 kits (MSD, Rockville, MD). IgE was measured with Invitrogen human IgE ELISA kit, and periostin was assayed with the Invitrogen human periostin ELISA kit (ThermoFisher Scientific, Frederick, MD). For statistical analysis, ANOVA was utilized to compare PN and control patients using log-transformed data. For between-markers multiplicity adjustment, adjusted p-value was calculated with a Benjamini-Hochberg procedure with the significance threshold at 0.05.

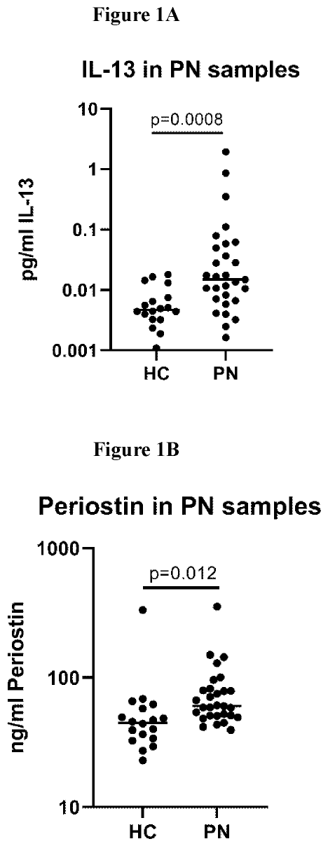

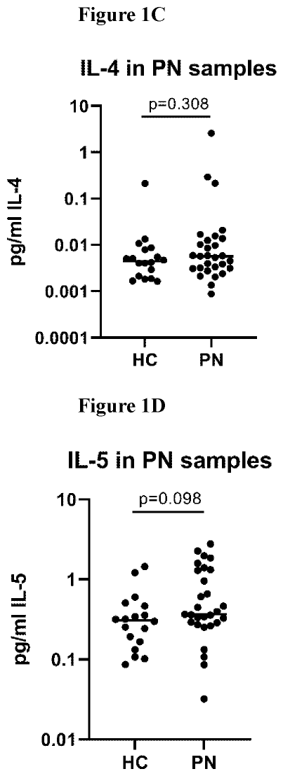

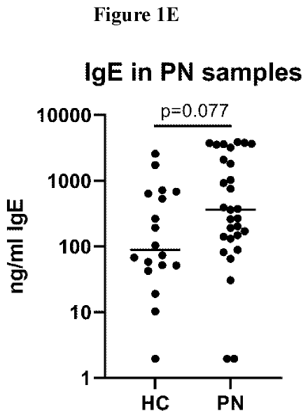

As shown in FIG. 1A, plasma IL-13 levels were significantly elevated (a 4-fold change with p=0.002) in PN samples (n=29) compared to HC samples (n=18). The elevated IL-13 levels in PN patient samples suggest an over-expression of IL-13 in PN patients, and IL-13 might be a key contributor to the disease pathogenesis. An IL-13 inhibitor (e.g., lebrikizumab) might be beneficial for the PN patients.

Periostin, a known biomarker associated with IL-13, is also elevated in PN patients compared to HC (FIG. 1B). IL-4, IL-5, and IgE are not significantly elevated in PN patients compared to HC (see Figures IC-1E).

### Example 2. A Global Phase 3, Multicenter, Randomized, Double-Blind, Placebo-Controlled Trial to Evaluate the Efficacy and Safety of Lebrikizumab Compared to Placebo in Adult Participants with Moderate-to-Severe Prurigo Nodularis

This is a global Phase 3, multicenter, randomized, double-blind, placebo-controlled, multiple period study to evaluate the efficacy and safety of lebrikizumab in adult participants with moderate-to-severe prurigo nodularis.

### Objectives and Endpoints

The primary objective of this study is to demonstrate the superiority of lebrikizumab versus placebo in itch response in the treatment of participants with prurigo nodularis. The primary endpoint is the proportion of participants with an improvement of ≥4 points from baseline in Itch NRS at Week 16.

The main secondary objectives include: (1) to demonstrate the superiority of lebrikizumab versus placebo in both itch response and skin efficacy in the treatment of participants with prurigo nodularis; and (2) to assess meaningful aspects of treatment benefit of lebrikizumab compared to placebo during the 16-week period. Accordingly, the main secondary endpoints include: (1) the proportion of participants that achieve both an IGA PN-S score of 0 or 1 with ≥2-point improvement from baseline and an improvement of ≥4 points from baseline in Itch NRS at Week 16; (2) the proportion of participants that achieve an IGA PN-S score of 0 or 1 with ≥2-point improvement from baseline at Week 16; (3) the proportion of participants who achieve a ≥1.5-point improvement in the frequency of nighttime awakenings due to itch as measured by DSS at Week 16, in participants with score of ≥1.5 or more points at baseline; (4) the proportion of participants with an improvement of ≥4 points from baseline in Itch NRS at Week 4; (5) the proportion of participants with an improvement of ≥4 points from baseline in Itch NRS at Week 2; (6) the proportion of participants with an improvement of ≥4 points from baseline in Itch NRS at Week 12; (7) the proportion of participants with an improvement of ≥4 points from baseline in Skin Pain NRS at Week 16 in participants with a Skin Pain NRS ≥4 at baseline.

Other secondary objectives include: (1) to assess meaningful aspects of treatment benefit of lebrikizumab compared to placebo during the 16-week treatment period; (2) to assess whether lebrikizumab is superior to placebo with respect to pruriginous lesions during the 16-week treatment period; (3) to evaluate the pharmacokinetics of lebrikizumab in participants with PN. Other secondary endpoints include: (1) the proportion of participants that achieve an IGA PN-A score of 0 or 1 (in those with a baseline IGA PN-A score of ≥3) by visit through Week 16; (2) change from baseline in PGI-S-PN by visit through Week 16; (3) time to onset of pruritus improvement as measured by an improvement in Itch NRS by ≥4 points from baseline during 16-week treatment period; (4) the proportion of participants that achieve an IGA PN-S score of 0 or 1 with ≥2-point improvement from baseline by visit through Week 16; (5) the proportion of participants with an improvement of ≥4 points from baseline in Itch NRS by visit in participants with an Itch NRS ≥7 at baseline; (6) the proportion of participants that achieve both an IGA PN-S score of 0 or 1 with ≥2-point improvement from baseline and an improvement of ≥4 points from baseline in Itch NRS by visit (i.e., a subject would be a responder if he/she meets both criteria jointly); (7) the proportion of participants who achieve a 1.5 or more-point improvement in the frequency of nighttime awakenings due to itch by visit in participants with score of ≥1.5 or more points at baseline; (8) the proportion of participants with an improvement of ≥4 points from baseline in Skin Pain NRS by visit in participants with a Skin Pain NRS ≥4 points at baseline; (9) the proportion of participants achieving ≥4-point improvement in DLQI from baseline to Week 16 in participants with a DLQI score ≥4 points at baseline; (10) the percentage of pruriginous lesions with excoriations/crusts (PAS item 7a) at each visit through Week 16; (11) the percentage of healed prurigo lesions (PAS item 7b) at each visit through Week 16; (12) change from baseline in number of lesions in representative area (PAS item 5) by visit through Week 16; (13) the steady-state concentration of lebrikizumab.

The exploratory objectives and endpoints include, but not be limited to, evaluations of the following at various study time points: (1) change from baseline in Itch NRS by visit through Week 16; (2) change from baseline in Skin Pain NRS by visit through Week 16; (3) change from baseline in sleep disturbance as assessed by the frequency of nighttime awakenings due to itch by visit through Week 16; (4) change from baseline in DLQI by visit through Week 16; (5) change from baseline in PROMIS depression measure by visit through Week 16; (6) change from baseline in PROMIS anxiety measure by visit through Week 16; (7) change from baseline in EQ5D-5L by visit through Week 16; (8) the proportion of participants achieving ≥4-point improvement in DLQI from baseline by visit in participants with a DLQI score ≥4 at baseline; (9) the proportion of participants with Itch NRS <2 at Week 16.

### Patient Population

Enough participants are screened to achieve approximately 300 randomly assigned to study intervention. Participants are randomized in 2:1 ratio to receive either lebrikizumab or placebo: approximately 200 in the lebrikizumab group and approximately 100 in the placebo group.

Inclusion Criteria: Participants are eligible to be included in the study only if all of the following criteria apply:
1. Participants≥18 years of age at the time of screening.
2. Have moderate-to-severe PN, as determined by all of the following:
   a. Clinical diagnosis of PN for at least 6 months;
   b. PN lesions on upper limbs, trunk, and/or lower limbs, at least 20 nodules on the entire body with a bilateral distribution;
   c. IGA PN-S score≥3 (based on the IGA scale ranging from 0 to 4, in which 3 is moderate and 4 is severe) at both the screening and baseline visits;
   d. Itch (pruritus) defined as a score of ≥7 (average score for a minimum of 4 of the 7 days immediately prior to randomization) at baseline visit on the Itch NRS.
3. History of inadequate response to treatment with topical medications; or determination that topical treatments are otherwise medically inadvisable.
4. Contraceptive use by men or women should be consistent with local regulations regarding the methods of contraception for those participating in clinical studies.
5. Capable of giving signed informed consent.

Exclusion Criteria: Participants are excluded from the study if any of the following criteria apply:
1. Chronic pruritus resulting from another active condition other than PN, such as but not limited to scabies, lichen simplex chronicus, psoriasis, atopic dermatitis (AD, history of AD is not reason for exclusion), contact dermatitis, acne, folliculitis, lichen planus, habitual picking/excoriation disorder, sporotrichosis, bullous autoimmune disease, end-stage renal disease, cholestatic liver disease (for example, primary biliary cirrhosis), diabetes mellitus, or thyroid disease that is not adequately treated per standard of care. If a patient has a positive history of AD, the investigator must ensure during the full exam that there are no active AD lesions. If the patient has any active lesions, the patient is excluded from the study.
2. Unilateral lesions of prurigo (for example, only 1 arm affected).
3. History of or current confounding skin condition (for example, Netherton syndrome, cutaneous T-cell lymphoma [mycosis fungoides or Sezary syndrome], chronic actinic dermatitis, dermatitis herpetiformis) or presence of skin comorbidities that may interfere with study assessments.
4. Neuropathic and psychogenic pruritus such as, but not limited to, notalgia paresthetica, brachioradial pruritus, small fiber neuropathy, diabetic neuropathy (and not diabetes), skin picking syndrome, or delusional parasitosis.
5. Are, in the judgment of the investigator, actively suicidal and therefore deemed to be at significant risk of suicide.
6. Have answered “yes” to either Question 4 or Question 5 on the “Suicidal Ideation” portion of the Columbia Suicide Severity Rating Scale (C-SSRS) or have answered “yes” to any of the suicide-related behaviors on the “suicidal behavior” portion of the C-SSRS, and the ideation or behavior occurred within the past month.
7. Participants with clinically significant systemic disease, including but not limited to renal, neuropsychiatric, cardiovascular, or hepatic as deemed by the principal investigator that would impact data or ability of subject to participate.
8. Uncontrolled chronic pulmonary disease that might require bursts of oral corticosteroids, e.g., co-morbid severe uncontrolled asthma, COPD or chronic bronchitis (a history of ≥1 exacerbation within the last 12 months requiring systemic [oral and/or parenteral] corticosteroid treatment or hospitalization for >24 hours).
9. Have known liver cirrhosis and/or chronic hepatitis of any etiology.
10. History of malignancy, including mycosis fungoides, within 5 years before the screening visit, except completely treated in situ carcinoma of the cervix, completely treated and resolved non-metastatic squamous or basal cell carcinoma of the skin with no evidence of recurrence in the past 12 weeks.
11. Severe concomitant illness(es) that in the Investigator's judgment would adversely affect the patient's participation in the study.
12. Any other medical or psychological condition that in the opinion of the Investigator may suggest a new and/or insufficiently understood disease, may present an unreasonable risk to the study patient because of his/her participation in this clinical trial, may make patient's participation unreliable, or may interfere with study assessments.
13. Pregnant or breastfeeding women, or women planning to become pregnant or breastfeed during the study.
14. Have fibromyalgia or other chronic pain condition that would confound evaluation of the patient.
15. Have any of the following infections:
   i. Have superficial skin infections treated either locally or systemically within 1 week of the baseline visit (can be rescreened after infection resolves).
   ii. Have a current or recent acute, active infection. For at least 30 days prior to screening and until the randomization visit, participants must have no symptoms and/or signs of confirmed or suspected infection, and must have completed any appropriate anti-infective treatment (can be rescreened after infection resolves).
   iii. Have had any of the following types of infection within 3 months prior to screening or develops any of these infections before the randomization visit:
   a) Serious infection (requiring hospitalization, and/or intravenous or equivalent oral antibiotic treatment);
   b) Opportunistic (as defined in Winthrop et al. 2015), for example, Herpes zoster is considered active and ongoing until all vesicles are dry and crusted over;
   c) Chronic infection (duration of symptoms, signs, and/or treatment of 6 weeks or longer);
   d) Recurring infection (including, but not limited to, herpes zoster, recurring cellulitis, chronic osteomyelitis). Participants with only recurrent, mild and uncomplicated orolabial herpes, or genital herpes, or both may be discussed with the sponsor's designated medical monitor for possible exemption from this exclusion criterion.
   iv. Have human immunodeficiency virus (HIV) infection.
   v. Have a current infection with hepatitis B virus (HBV) (that is, positive for hepatitis B surface antigen and/or PCR positive for HBV DNA.
   vi. Have a current infection with hepatitis C virus (HCV) (that is, positive for HCV RNA).
   vii. Active tuberculosis (TB).
   viii. Have received a Bacillus Calmette-Guerin (BCG) vaccination or treatment within less than 4 weeks before randomization; or received any other live vaccine (that is, live attenuated) within less than 4 weeks before randomization, or intend to receive a live vaccine during the study.
   ix. Diagnosed with active endoparasitic infections or at high risk of these infections.
16. Prior treatment with dupilumab or nemolizumab.
17. Treatment with any of the following agents within 4 weeks prior to the baseline visit:
   a. Immunosuppressive/immunomodulating drugs (e.g., systemic corticosteroids, cyclosporine, mycophenolate-mofetil, IFN-y, Janus kinase inhibitors, azathioprine, methotrexate, etc.);
   b. Phototherapy and photochemotherapy (PUVA).
18. Treatment with the following prior to the baseline visit:
   a. An investigational drug within 8 weeks or within 5 half-lives (if known), whichever is longer;
   b. B Cell-depleting biologics, including rituximab, within 6 months;
   c. Other biologics within 8 weeks or 2 half-lives, whichever is longer.
19. Use of prescription moisturizers within 7 days of the baseline visit.
20. Regular use (more than 2 visits per week) of a tanning booth/parlor within 4 weeks of the screening visit.
21. Use of systemic corticosteroids at average daily doses of ≥10 mg/day of prednisone or its equivalent, or use of variable doses of any systemic corticosteroids, within 4weeks prior to baseline.
22. Use of anti-pruritic therapies (e.g., capsaicin, ion channel blockers, cannabinoids, antihistaminic therapies) within 7 days of the baseline visit.
23. Are currently enrolled in any other clinical study involving an investigational product or any other type of medical research judged not to be scientifically or medically compatible with this study.
24. Participation in a prior lebrikizumab clinical study.
25. Have clinical laboratory test results at entry that are outside the normal reference range for the population or investigational site, or results with unacceptable deviations that are considered clinically significant by the investigator, and/or have any of the following specific abnormalities:
   a. Aspartate aminotransferase (AST) or alanine aminotransferase (ALT) ≥2× the ULN
   b. Total bilirubin ≥1.5× the ULN
   c. Total white blood cell (WBC) <2500 cells/μL
   d. Hemoglobin <9.0 g/dL
   e. Neutropenia (absolute neutrophil count) <1200 cells/μL
   f. Thrombocytopenia (platelets) <75,000 cells/μL
   g. eGFR <40 mL/min/1.73 m2 at Visit 1.
26. Have evidence of current or within 1 year a history of any substance use disorder(s) of any severity as defined by the DSM-V in the opinion of the investigator, excepting disorders of nicotine or caffeine use.
27. Use of cannabis or cannabinoids for the treatment of pruritus, pain, and AD (the recreational use of cannabis and cannabinoid is permitted).

### Study Drug

Pharmaceutical compositions containing 125 mg/mL lebrikizumab or placebo are supplied as sterile pre-filled syringes with a pre-assembled needle safety device (PFS-NSD) for subcutaneous administration to the patients. Lebrikizumab sequences are provided in Table 1. The placebo solution is identical in appearance and volume to the active solution except that it does not contain lebrikizumab.

### Study Design

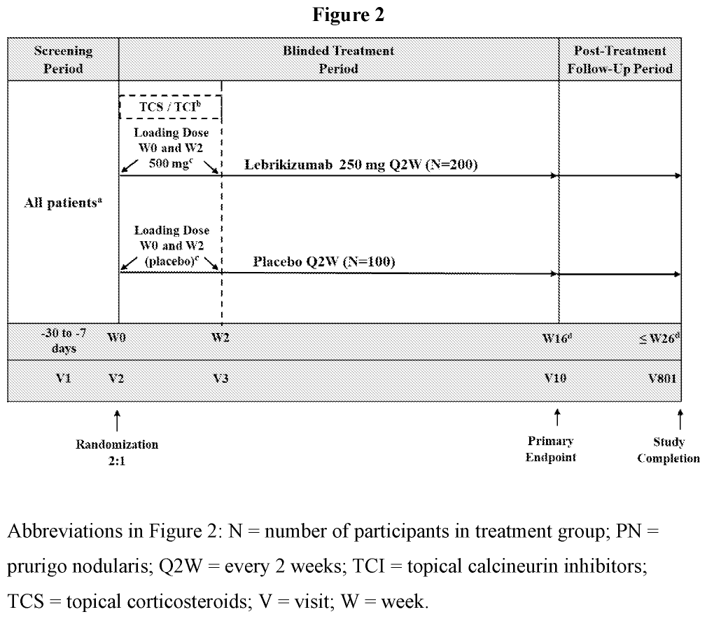

The study design of this trial is shown in FIG. 2.

At baseline (Week 0), participants who meet the study eligibility criteria are randomly assigned in a 2:1 ratio to lebrikizumab 250 mg Q2W (loading dose of 500 mg given at Week 0 and Week 2) or matching placebo by subcutaneous (SC) injection. Randomization are stratified based on geographic region (United States versus Europe versus the rest of world) and disease severity (IGA PN-S, 3 versus 4). Participants are allowed to use moisturizers.

During the Blinded Treatment Period, participants who require use of low-to mid-potency TCS or TCI may do so only during the first 2 weeks of the period (≤3 consecutive days and no more than 5 cumulative days). Such participants are not considered treatment failures (non-responders) in the efficacy analyses. During the Blinded Treatment Period, the use of systemic treatments for PN is prohibited and the use of topical treatments for PN is prohibited after Week 2.

If participants experience intolerable PN symptoms and require rescue treatment, they should preferably be started on topical treatments (for example, low-to mid-potency TCS) prior to instituting high potency TCS or systemic treatments for PN symptoms. If high-potency TCS or systemic rescue treatments are required, participant must be permanently discontinued from blinded study drug. The patient continues to attend all study visits through Week 16 and be assessed for safety and efficacy according to the schedule of events. Participants requiring use of rescue treatment (i.e., any TCS/TCI or systemic treatment, regardless of the indication for use) after Week 2 are considered treatment failures (non-responders) in the efficacy analyses during the Blinded Treatment Period.

The primary efficacy endpoints are assessed at Week 16. Efficacy and health outcomes/quality of life are measured using Itch NRS, IGA PN-S, IGA PN-A, Skin Pain NRS, nighttime awakenings DSS, PGI-S-PN, DLQI, PAS, PROMIS Anxiety and Depressive Symptoms, and EuroQol-5D (EQ-5D-5L, European Quality of Life-5 Dimensions-5 Levels).

Safety is assessed by monitoring AE, serum chemistry, hematology and urinalysis laboratory testing, physical examination, pulse and blood pressure, and the C-SSRS, which assesses suicide risk. Additionally, serum samples are collected to assess PK and immunogenicity.

Statistical analyses are performed for the primary and secondary endpoints.

1. A method of treating prurigo nodularis or reducing pruritus associated with prurigo nodularis in a patient in need thereof, the method comprising administering to the patient a therapeutically effective amount of an IL-13 inhibitor.

2. The method of claim 1, wherein the IL-13 inhibitor is an anti-IL-13 antibody.

3. The method of claim 2, wherein the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

4. The method of claim 2, wherein the anti-IL-13 antibody comprises a VH comprising SEQ ID NO: 7, and a VL comprising SEQ ID NO: 8.

5. The method of claim 2, wherein the anti-IL-13 antibody comprises a heavy chain comprising SEQ ID NO: 9, and a light chain comprising SEQ ID NO: 10.

6. The method of claim 2, wherein the anti-IL-13 antibody is lebrikizumab.

7. The method of claim 2, wherein the anti-IL-13 antibody is administered subcutaneously to the patient.

8. The method of claim 2, wherein the anti-IL-13 antibody is administered at a dose of 250 mg to 500 mg.

9. The method of claim 2, wherein the anti-IL-13 antibody is administered subcutaneously to the patient at a dose of 250 mg once every two weeks.

10. The method of claim 9, the patient is further treated with a loading dose of 500 mg of the anti-IL-13 antibody at week 0 (baseline) and week 2.

11. (canceled)

12. The method of claim 2, wherein the patient is treated with the anti-IL-13 antibody for a period of about 16 to about 24 weeks.

13. The method of claim 12, wherein the patient is treated with the anti-IL-13antibody for a period of about 16 weeks.

14. The method of claim 12, wherein the patient is treated with the anti-IL-13antibody for a period of about 24 weeks.

15. The method of claim 2, further comprising determining the Itch Numerical Rating Scale (NRS) score of the patient before and after the treatment.

16. The method of claim 2, further comprising determining one or more of the following characteristics of the patient: IGA PN-S, IGA PN-A, Skin Pain NRS, nighttime awakenings DSS, PGI-S-PN, DLQI, PAS, PROMIS Anxiety and Depressive Symptoms, and EuroQol-5D (EQ-5D-5L) before and after the treatment.

17. The method of claim 2, wherein the anti-IL-13 antibody is administered to the patient using a subcutaneous administration device.

18. The method of claim 17, wherein the subcutaneous administration device is selected from a prefilled syringe, disposable pen injection device, microneedle device, microinfuser device, needle-free injection device, or autoinjector device.

19. The method of claim 2, wherein the patient has moderate to severe prurigo nodularis.

20. The method of claim 19, wherein the patient has (1) a clinical diagnosis of PN for at least 6 months; (2) PN lesions on upper limbs, trunk, and/or lower limbs, at least 20 nodules on the entire body with a bilateral distribution; (3) a IGA PN-S score ≥3 at baseline; and/or (4) an Itch NRS score ≥7 at baseline.

21. The method of claim 2, wherein the patient has inadequate response to topical corticosteroids or topical calcineurin inhibitors, or topical corticosteroids or topical calcineurin inhibitors are medically inadvisable for the patient.

22. The method of claim 2, wherein the patient is aged 18 years or older.

23.-30. (canceled)
